(19) United States
(12) Patent Application Publication
(10) Pub. No.: US 2003/0195255 A1
(43) Pub. Date: Oct. 16, 2003

(54) Method for treating hepatic encephalopathies

(76) Inventor: Marshall L. Summar, Brentwood, TN (US)

Correspondence Address: REED SMITH LLP, 2500 ONE LIBERTY PLACE, 1650 MARKET STREET, PHILADELPHIA, PA, 19103, US

(21) Appl. No.: 10/122,445

(22) Filed: Apr. 12, 2002

(51) Int. Cl. A61K 31/192

(52) U.S. Cl. 514/570

(57) ABSTRACT

A method for using phenyl butyrate compounds, their salts, derivatives and metabolites to treat chronic hepatic encephalopathies. Treatment according to the invention can arrest and even reverse the loss of mental function associated with chronic hepatic encephalopathies.

### BACKGROUND OF THE INVENTION

This invention relates to the treatment of a class of brain disorders known as chronic hepatic encephalopathies. Hepatic encephalopathies are characterized by a progressive loss of brain and mental function, and are associated with disorders of liver function.

Liver disorders that can be associated with hepatic encephalopathies vary widely in their causation and clinical presentation. Hepatitis, cirrhosis, drug or alcohol abuse, and a variety of other disorders can be associated with hepatic encephalopathies. Hepatic encephalopathies can also result from physical disruption of metabolite delivery to the liver.

The loss of mental function associated with hepatic encephalopathies can be severe. Eventually, patients can lose their ability to carry out ordinary life functions, or even to recognize close relatives. The emotional toll taken by this disorder is heavy, as is the financial burden that it imposes on families and the community.

Phenyl butyrate and its metabolite phenyl acetate are known chemical entities. Sodium phenyl butyrate has been approved for use in the United States to treat disorders of urea cycle metabolism, and is sold under the trademark Buphenyl for that purpose. It has also been reported that certain of this class of components is effective as an anticancer agent (See, U.S. Pat. No. 6,037,376), and as an anti-viral (See, U.S. Pat. Nos. 5,877,213 and 5,710,178).

### SUMMARY OF THE INVENTION

According to the present invention, phenyl butyrate compounds, their salts, derivatives and metabolites are used to treat chronic hepatic encephalopathies. Treatment according to this invention can arrest and even reverse the loss of mental function associated with chronic hepatic encephalopathies.

In the practice of this invention, phenyl butyrate compounds, their salts, derivatives and metabolites are administered in an amount effective to achieve an optimum clinical result.

### DETAILED DESCRIPTION OF THE INVENTION

Sodium phenyl butyrate is conveniently available in a commercial preparation known as Buphenyl, sold by Ucyclid Pharma, of Scottsdale, Ariz. Buphenyl is prepared for oral delivery in tablet form.

Other related compounds which are useful in the current invention are the salts, derivatives and metabolites of phenyl butyrate. These are well known in the art.

U.S. Pat. No. 4,456,942 discloses a group of phenyl acetate derivatives useful in the present invention. These compounds may be described by the following formula:

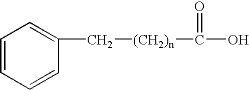

where n is 2, 4, 6 or 8.

Another group of compounds useful in the present invention is disclosed in U.S. Pat. No. 5,968,979, which describes phenylalkanoic esters of glycerol according to the following formula:

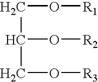

where R₁, R₂ and R₃ are independently, H

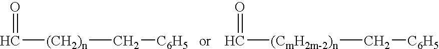

where n is 0 or an even number from 2-24 and m is an even number from 2-24, provided that at least one of R₁, R₂ and R₃ is not H. Glyceryl-tri (4 phenyl butyrate) is an example of such a compound.

Other compounds useful in the method of this invention include phenylacetic acid, its salts (especially sodium salts), halogenated analogs, and alkyl substituted analogs. Specific examples include sodium phenyl acetate and napthyl acetate.

The use of sodium phenyl butyrate to treat chronic hepatic encephalopathy was demonstrated with a group of six patients. Each of these patients suffered from moderate to severe chronic hepatic encephalopathy, and had lost significant mental function as a consequence of the disorder.

The patients in this group suffered from a variety of liver diseases, including Hepatitis C, cirrhosis, and damage caused by drug abuse. At least one patient suffered from a combination of these disorders.

Each patient was given 6 gm/m²/day of sodium phenyl butyrate, divided into three doses. This was done for seven days, during which time the patient's blood chemistry and overall health was monitored and evaluated.

At the end of the seven day regimen, the patients' mental state was reported.

One patient who had suffered significant impairment regained the ability to balance her checkbook, and her family reported a significant improvement in her ability to communicate with others. Another seriously impaired patient regained the ability to drive his car. All patients reported a recovery of mental function, although this benefit was reported to decrease after the use of the drug was terminated.

The improvement in mental function achieved by the method of the present invention has been apparent, as is reported above. Other techniques for measuring improved mental function, such as the PHES score, and auditory nerve conduction studies can be used to demonstrate the effectiveness of this invention.

The dose used in this study proved to be efficacious. However, the dose used in clinical practice will necessarily be adjusted in accordance with the good clinical judgment of the physician. Factors that will be ordinarily considered in this regard include the patient's tolerance for the drug (some of which are known to be difficult to take orally), the severity of the patient's hepatic encephalopathy, the patient's ability to absorb the drug, the patient's total sodium intake, and other factors. Occasionally, it may be necessary to measure the patient's blood levels of sodium phenyl butyrate. Such ongoing clinical observation and dosage adjustment are commonplace in good medical practice.

In the above described experiment, the method of this invention was carried out by administering the drug orally. It may be desirable in some circumstances to administer the drug parentally. Some compounds useful in the practice of this invention may be more effective when administered parentally, and others suffer from unpleasant side effects when admitted orally. Intravenous administration is particularly suitable for comatose patients who can be awakened from the comatose state by this method. Sodium phenyl acetate is well suited to parental administration, especially in combination with sodium benzoate. A suitable regimen consists of an initial loading dose and regular additional doses. For example, in infants, a loading dose of about 200-300 mg/kg (preferably about 250 mg/kg) given over 1-2 hours, followed by daily administration of about 200-300 mg/kg (preferably about 250 mg/kg), divided in three, is effective. In adults, a loading and daily dose of about 3.0 to about 8.0 g/m² (preferably about 5 to about 6 g/m²) is effective.

Generally, the orally administered daily dose of sodium phenyl butyrate used in this invention is between about 3 and about 12 g/m². More commonly, the daily dose will be between about 6 and about 9 g/m².

1. A method of treating hepatic encephalopathy, comprising administering to a person exhibiting hepatic encephalopathy a therapeutically effective amount of at least one phenyl butyrate compound or a salt, derivative or metabolite of phenyl butyrate in a pharmaceutically acceptable vehicle.

2. The method of claim 1, wherein the phenyl butyrate compound, or a salt, derivative or metabolite of phenyl butyrate is administered orally.

3. The method of claim 2, wherein the phenyl butyrate compound, or a salt, derivative or metabolite of phenyl butyrate is administered in an amount from about 3 to about 12 g/m²/day.

4. The method of claim 2, wherein the phenyl butyrate compound, or a salt, derivative or metabolite of phenyl butyrate is administered in an amount from about 6 to about 9 g/m²/day.

5. The method of claims 2, 3 or 4 wherein the compound is sodium phenyl butyrate.

6. The method of claims 2, 3 or 4 wherein the compound is glyceryl-tri (4 phenyl butyrate).

7. The method of claim 1, wherein the phenyl butyrate compound or a salt, derivative or metabolite of phenyl butyrate is delivered parentally.

8. The method of claim 7, wherein the phenyl butyrate compound or a salt, derivative or metabolite of phenyl butyrate is administered to an adult in an amount from about 3 to about 8 g/m²/day.

9. The method of claim 8, when an initial loading dose of from about 3 to about 8 g/m² is additionally administered to the person.

10. The method of claim 9, wherein the phenyl butyrate compound or a salt, derivative or metabolite of phenyl butyrate is administered in amount from about 5 to about 6 g/m²/day.

11. The method of claim 10, wherein an initial loading dose of from about 5 to about 6 g/m² is additionally administered to the person.

12. The method of claims 7, 8, 9, 10 or 11, wherein the compound is sodium phenyl butyrate.

13. The method of claims 7, 8, 9, 10 or 11, wherein the compound is sodium phenyl acetate.

14. The method of claim 13 where sodium benzoate is additionally administered to the person.
